(19) United States
(12) Patent Application Publication
(10) Pub. No.: US 2013/0131757 A1
(43) Pub. Date: May 23, 2013

(54) MUSCLE FATIGUE METER

(71) Applicants: Michael Robert Duncan, Lane Cove (AU); Simon Geoffrey Parker, Ryde (AU)

(72) Inventors: Michael Robert Duncan, Lane Cove (AU); Simon Geoffrey Parker, Ryde (AU)

(21) Appl. No.: 13/664,663

(22) Filed: Oct. 31, 2012

Related U.S. Application Data
(63) Continuation of application No. 12/464,699, filed on May 12, 2009, now abandoned.
Application No. 12/464,699 is a continuation of application No. 10/344,552, filed on Mar. 25, 2003, now abandoned.

(30) Foreign Application Priority Data
Aug. 14, 2000 (AU) PQ9413
Aug. 15, 2000 (AU) PQ9441

(51) Int. Cl. A61N 1/36

(52) U.S. Cl. CPC A61N 1/36003
USPC 607/49

(57) ABSTRACT

A functional electrical stimulation system for moving at least a portion of a body of a subject, e.g., legs. Such a system includes: a stimulator that can provide a plurality of sets of functional electrical stimulation to the legs, one or more transducers that outputs signals representative of the movement made by the legs in response to the functional electrical stimulation provided thereto, and a controller. The controller compares the output signals of the transducers resulting from the provision of two or more substantially equivalent sets of electrical stimulation to the legs and provide an output indicative of variation in the movement. The controller also, e.g., can compare the level of electrical stimulation output by the stimulator to achieve an equivalent movement of the legs so as to recognize an increase in such stimulation, where an increase can be indicative of fatigue in the leg muscles.

### FIELD OF THE INVENTION

The present invention relates to a functional electrical stimulation (FES) system and method of using such a system. More particularly, the invention relates to a device and method for determining the presence of fatigue in muscles receiving stimulation from a functional electrical stimulation system.

### BACKGROUND OF THE INVENTION

Functional electrical stimulation (FES) systems have been developed using electronic body worn equipment which generates and delivers electrical impulses to the body to control muscle movement.

Functional electrical stimulation (FES) systems are seen to have particular future application in providing persons suffering from spinal cord injury or deficiency, such as paraplegia, with a capacity to make controlled movements of their dysfunctional limbs.

Functional electrical stimulation systems use electronics to generate electrical impulses. These impulses are then delivered to nerves or muscles of a subject via electrodes to stimulate movement of the muscles that are otherwise dysfunctional. In order for useful and controlled movements of limbs to be achieved several muscles must usually be operated in concert. This is normally achieved by an algorithm executed under the control of the FES system to deliver a pattern of stimulation impulses.

Just as a person's functional muscles can become fatigued through constant or excessive use, a persons dysfunctional muscles can also become fatigued if subjected to repeated functional electrical stimulation (FES). Due to the fact that in FES systems stimulation is applied to the subject's muscles based on the desired movement required, actual muscle fatigue is not perceptible to the subject and as such the subject is not directly aware of the possible state of fatigue which their muscles may be under. There is, accordingly, potential for the subject's muscles to be stimulated beyond an appropriate level of fatigue thereby raising the possibility of serious injury being caused to the subject's muscles due to the subjects muscles failing to perform the desired task and the subject falling.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention before the priority date of each claim of this application.

### SUMMARY OF THE INVENTION

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

According to a first aspect, the present invention is a functional electrical stimulation system for moving at least a portion of a body of a subject, the system comprising:

a stimulating means adapted to provide a plurality of sets of functional electrical stimulation to a portion of a subject's body;

a monitoring means that outputs signals representative of the movement made by said portion of the subject's body in response to the functional electrical stimulation provided thereto; and

a control means that receives and processes the signals output by the monitoring means;

wherein the control means including a comparator means adapted to compare the output signals of the monitoring means resulting from the provision of two or more substantially equivalent sets of electrical stimulation to said portion of the subject's body and provide an output indicative of variation in the movement.

In this aspect, the monitoring means outputs signals representative of the magnitude of movement of said body portion.

In a preferred embodiment, the sets of electrical stimulation are preferably equivalent.

In one embodiment, the monitoring means outputs signals representative of the movement of a subject's limb that has been electrically stimulated by the stimulating means. In a preferred embodiment, the monitoring means outputs signals representative of the movement of the subject's leg or legs in response to electrical stimulation of one or both legs. In a preferred embodiment, the monitoring means outputs signals representative of the magnitude of movement of a leg caused by electrical stimulation provided to the nerves or muscles of that leg.

The monitoring means preferably includes at least one transducer mountable to said portion of the body of said subject. The transducer is preferably mountable to the body at a position suitable to monitor the movement of the body portion made in response to the electrical stimulation. When the legs are being stimulated, the at least one transducer is preferably mountable to one of the legs of the subject. A single transducer can be mounted to each leg of the subject or a plurality of transducers can be to mounted to each leg. The at least one transducer preferably converts detected limb movement into output signals. In a further embodiment, the at least one transducer can be implantable within the subject.

The control means preferably includes a memory means that stores the measured magnitude of movement of a stimulated limb following the provision of a set of electrical stimulation thereto. The memory means can preferably store a plurality of measured limb movement magnitudes.

The comparator means can preferably make a first comparison of the measured magnitude of limb movement from each of a sequence of sets of electrical stimulation provided to that limb. For example, if a leg receives a first set of electrical stimulation from the stimulating means so as to cause the lower leg to undergo flexion relative to the thigh, the monitoring means preferably receives and stores the magnitude of movement of the lower leg that has resulted from the stimulation. If an equivalent stimulation set is subsequently provided to again cause flexion of that lower leg, the memory means receives and stores the magnitude of movement that has resulted from the subsequent stimulation. The memory means can continue to store lower leg movement magnitudes for each equivalent stimulation.

The comparator means in mucking the first comparison can then compare the stored movement magnitudes and measure whether there is any variation in movement magnitudes over time. A decrease in movement magnitude may be due to fatigue within the stimulated muscles of that limb. If the movement magnitude falls below a preset level in comparison to that achieved from a selected earlier stimulation, the control means can output a warning signal that the stimulated muscle is becoming fatigued.

In one embodiment, the control means can output a warning signal when the movement magnitude falls to below about 98% of the movement magnitude resulting from the selected earlier stimulation, more preferably below about 80% of the movement magnitude resulting from the selected earlier stimulation, and still more preferably below about 75% of the movement magnitude resulting from the selected earlier stimulation.

The warning signal can be output to a warning means that provides an indication to the subject of muscle fatigue in said body portion. Such a warning may lead to the subject simply resting the limb or muscles that have been receiving stimulation. The indication can be provided to the subject through visual and/or audible indication. Visual indication may include a warning light or written message on a screen display, such as a LCD display.

Instead of or in addition to providing a warning to the subject, the warning signal can be provided to an override means which prevents muscle stimulation to the fatigued limb. The override means can prevent muscle stimulation for a predetermined that is considered sufficient to allow the muscles of the limb to recover to a level sufficient to again be stimulated.

Where the functional electrical stimulation system is being used to stimulate more than one muscle or more than one limb, the override means can be adapted to only prevent stimulation to fatigued muscles and not muscles that are capable of receiving stimulation by the system. In such instances where a group of muscles are to be stimulated and one or more of such muscles may be fatigued, the override system can prevent such stimulation from occurring thereby protecting the fatigued muscle from further exhaustion.

In a further embodiment of the first aspect, the control means can modify the level of electrical stimulation provided to the subject's muscles in response to the first comparison of limb movement magnitude determined by the comparator means. If the comparator means records a reduction in movement magnitude, the control means can instruct the stimulating means to increase the electrical stimulation provided to the muscle when the control means next instructs the stimulating means to stimulate the muscle. The magnitude of increase can be predetermined or set by the control means.

The stimulating means preferably has a maximum stimulation level which prevents stimulation beyond that level.

On increasing the stimulation, the control means again measures limb movement magnitude. The comparator means then undertakes a second comparison of limb movement arising from the increased stimulation to that limb. The control means is therefore able to determine if the increase in stimulation has been sufficient to restore required limb movement magnitude. If the increase in stimulation level has been sufficient, subsequent stimulations can remain at that increased level. If the limb movement magnitude falls, the control means can again increase the level of stimulation when the control means again instructs the stimulating means to stimulate the muscle.

In a still further embodiment, the comparator means can undertake a third comparison of required stimulation level to achieve an equivalent limb movement magnitude over time on provision of subsequent sets of electrical stimulation to said body portion. In this embodiment, the required level of stimulation would be provided to the comparator means by the control means and the measured limb movement magnitudes (so as to ensure that the limb movement magnitudes are about the same) would be measured by the transducers on the limb.

While the above description has focussed on increasing stimulation in response to muscle fatigue, it will be appreciated that the system can decrease the level of stimulation in response to a noted decrease in muscle fatigue.

The second and third comparisons undertaken by the comparator means provide another means of monitoring muscle fatigue of a stimulated muscle. By comparing the level of required stimulation to achieve a desired particular magnitude of limb movement over time, it is possible to provide a measure of the degree of muscle fatigue in the muscle. Once the required level of muscle stimulation is at a predetermined level that is greater than that required at a selected first stimulation, the control means can output a warning signal that the stimulated muscle is becoming fatigued. The warning system can be identical to that described above.

In one embodiment, the control means can output a warning signal when the stimulation level is at least about 10% greater, more preferably is at least 25% greater, and still more preferably is at least about 50% greater, than the stimulation level required for the selected first stimulation.

The control means can also output signals to the stimulating means instructing the stimulating means to provide electrical stimulation to said body portion of the subject.

The signals output by the transducer and the signals output by the control means to the stimulating means can comprise electrical or optical signals.

The monitoring means, or componentry thereof, can be carried by the subject. The monitoring means can be carried in a harness or clothing worn by the subject. In another embodiment, the monitoring means may be strapped to the body of the subject. In an alternative embodiment, the monitoring means or componentry thereof can be implantable within the subject.

In one embodiment, the stimulating means comprises a stimulator and one or more electrodes electrically connected to the output of the stimulator. The stimulator can be carried by the subject. The stimulator may be carried in a harness or clothing worn by the subject. In another embodiment, the stimulator may be strapped to the subject. In an alternative embodiment the stimulator or componentry thereof can be implantable within the subject. The electrical leads extending from the stimulator to the electrodes can be totally implantable within the subject or carried externally on the body of the subject. Where the stimulators is implanted within the subject, the system can include an external controller adapted to provide a means of delivering control signals and/or power to the stimulating means. The external controller can communicate with the implanted stimulating means by way of radio frequency (RF) communication.

The electrodes can be surface mounted on the skin of the subject, can be percutaneous intramuscular electrodes that are implanted with a minimally invasive needle insertion procedure, or fully implanted electrodes. The electrodes can be taped or mounted on the skin of the subject in appropriate locations to assure suitable electrical stimulation is provided to said body portion. In another embodiment, the electrodes can be implantable at appropriate locations within said body portion.

Each set of electrical stimulation can comprise one or more individual stimulation impulses. In one embodiment, a set of stimulation impulses can comprise a predetermined sequence of individual impulses suitable for moving said body portion in a desired manner. For example, a sequence of stimulation impulses can be provided to a number of electrodes mounted to the leg of a subject to cause that leg to undertake a walking motion. In this case, it can be appreciated that the subjects other leg would also normally preferably receive a sequence of stimulation pulses but 180° out of phase to the impulses provided to the first mentioned leg. Other stimulation sequences can be envisaged for causing other desired movements of the legs, such as a cycling movement.

While stimulation impulses may be provided to both legs, fatigue monitoring using the system may only occur on one of the stimulated legs.

In a preferred embodiment, the system can be controlled by the subject. Control signals for the system can be provided by the subject adjusting the position or alignment of their body, such as their torso and/or head, in another embodiment, control signals can be provided by the subject adjusting the position or alignment of a device, such as a walking aid. Still further, the subject can control the system by utilising a hand-held or hand-operated device.

Movement of the torso, head and/or walking aid can be detected by transducers mounted or implanted within these structures. It is envisaged that a particular pre-set movement of the torso, for example, will lead to a pre-programmed desired pattern of stimulation to a limb so causing a desired movement of that limb. These transducers provide control signals to the control means which in turn instructs the stimulating means to stimulate the desired muscles of the subject.

In a preferred embodiment, the control means has an operating means. The operating means preferably comprises an activation and deactivation means. The activation and deactivation means preferably allows the subject to turn on and off the control means and the FES system when desired. Where the FES system is fully implanted, the activation and deactivation means is preferably controllable from outside the body, for example, by way of the external controller described above. In one embodiment, the activation and deactivation means can comprise a switch. Where the control means is implanted, the system preferably can still be operated through the skin of the subject. The operating means preferably incorporates a locking means to prevent inadvertent activation or deactivation of the system.

According to a second aspect, the present invention is a functional electrical stimulation system for moving at least a portion of a body of a subject, the system comprising:

a stimulating means adapted to provide a plurality of sets of functional electrical stimulation to said body portion;

a monitoring means that outputs signals representative of the movement made by said portion of the subject's body in response to the functional electrical stimulation provided thereto; and

a control means that receives and processes the signals output by the monitoring means during the provision of at least one of the sets of electrical stimulation and outputs suitable control signals to the stimulating means to vary the stimulation to said body portion to ensure the movement of said portion during that or a subsequent provision is at least substantially equivalent to that resulting from the provision of a selected set of electrical stimulation preceding said at least one set of electrical stimulation;

wherein the control means further includes a comparator means adapted to compare the control signals provided to the stimulating means resulting respectively from the provision of said at least one set of electrical stimulation and said at least one preceding set of electrical stimulation to said body portion and provide an output indicative of a variation between the respective control signals.

In this aspect, the monitoring means measures the magnitude of movement of said body portion.

In this further aspect, the stimulating means stimulates said body portion to ensure the movement of said body portion resulting from the provision of said at least one set of electrical stimulation is equivalent to the movement resulting from the selected preceding set of electrical stimulation.

In this further aspect, the stimulating means can have a maximum stimulation level.

Still further, the monitoring means can output signals representative of the magnitude of movement of a subjects limb that has been electrically stimulated by the stimulating means. In this embodiment, the monitoring means can output signals representative of the movement of the subject's leg or legs in response to electrical stimulation of one or both legs.

The monitoring means in this aspect can include at least one transducer mountable to said body portion of said subject, such as one of the legs of the subject. Alternatively, the at least one transducer can be implantable within the subject.

In this further aspect, the control means can include a memory means that stores the measured magnitude of movement of a stimulated limb following the provision of a set of electrical stimulation thereto. The memory means can store a plurality of measured limb movement magnitudes.

In one embodiment, if an increase in output by the stimulating means is required to cause the substantially equivalent movement magnitude of said body portion, this is taken by the control means to be due to fatigue of the muscles of said body portion

If the stimulation level output by the stimulating means during said subsequent provision of electrical stimulation increases above a pre-set level in comparison to that achieved from a selected earlier stimulation, the control means can output a warning signal that the muscles of said body portion are becoming fatigued.

The control means can output the warning signal when the electrical stimulation level is at least about 10% greater, more preferably is at least 25% greater, and still more preferably is at least about 50% greater, than the stimulation level required for the selected preceding set of electrical stimulation.

In this further aspect, the warning signal can be output to a warning means that provides an indication to the subject of muscle fatigue. The indication can be provided to the subject through a visual and/or audible means. The warning signal can still further be provided to an override means that prevents delivery of electrical stimulation to said body portion.

In the further aspect, the monitoring means can include a transducer mountable on said body portion. Still further, the stimulating means can comprise a stimulator and one or more electrodes mountable on said body portion and electrically connected to the output of the stimulator. Each set of electrical stimulation can comprise one or more individual stimulation pulses. Each set of electrical stimulation can also comprise a predetermined sequence of individual impulses suitable for moving said body portion in a desired manner.

According to a still further aspect, the present invention is a method of providing functional electrical stimulation to a portion of a subject's body, the method comprising the steps of:

providing at least one set of electrical stimulation to said body portion;

monitoring the movement of said body portion resulting from said first set of electrical stimulation and outputting storable signals representative of said movement;

providing at least one subsequent set of electrical stimulation to said body portion, said subsequent set being substantially equivalent to a selected one of said at least one set of electrical stimulation;

monitoring the movement of said body portion resulting from said subsequent set of electrical stimulation and outputting storable signals representative of said movement;

comparing the storable signals representative of the movement of said body portion resulting from said selected set of electrical stimulation with the storable signals representative of the movement of said body portion resulting from said subsequent set of electrical stimulation; and

outputting an indication signal indicative of variation in the movement resulting from the selected one and subsequent sets of electrical stimulation.

In a preferred embodiment, the method includes a step of storing the storable signals representative of movement made by the subjects body.

In one embodiment, the indication signal is used as a muscle fatigue warning signal if the magnitude of body movement falls below a predetermined level in comparison to that measured earlier, such as when the muscle was unfatigued. In one embodiment, the method can include a step of preventing electrical stimulation to at least said body portion if a muscle fatigue warning signal is generated.

In a further embodiment, the method includes a step of comparing limb movement magnitude resulting from a selected one set of electrical stimulation and that resulting from said subsequent set of electrical stimulation, where the subsequent set of electrical stimulation is different to that of the selected one set of electrical stimulation, in a preferred embodiment, the second electrical stimulation is at a level greater than that of the first electrical stimulation.

In another embodiment, the method includes a step of comparing the levels of sequential electrical stimulations that are required to achieve equivalent limb movement magnitude. By comparing the amount of electrical energy required to stimulate a muscle to perform a specific task when the muscle is not in a fatigued state with the amount of electrical energy required to stimulate the muscle after a time of stimulation, an indication of the amount of muscle fatigue can be gauged. This measurement/indication can use known patient specific data to determine the level of muscle fatigue of the patient's muscle or can use a model which follows known muscle fatigue patterns in patients undergoing electrical stimulation.

In another embodiment the method can be used to provide the subject with an estimate of current muscle strength which can be used to provide the subject with an indication of the amount of future activity possible prior to muscle fatigue being experienced. By understanding the capacity of the subject's muscles throughout a serious of movements the subject can further manage their movements to ensure that they stay within their limits and do not exert too much stress on their muscles.

According to a further aspect, the present invention is a method of providing functional electrical stimulation to a portion of a subject's body, the method comprising the steps of:

providing one or more sets of electrical stimulation to said body portion;

recording said one or more sets of stimulation in a recording means;

monitoring the movement of said body portion resulting from said one or more sets of electrical stimulation and outputting storable signals representative of said movement;

providing a subsequent set of electrical stimulation to said body portion and simultaneously monitoring and comparing the movement of said body portion resulting from said subsequent set of electrical stimulation to the storable signals of said one or more preceding sets of electrical stimulation;

adjusting the subsequent set of electrical stimulation to ensure the movement of said portion during the provision of the subsequent set is substantially equivalent to that resulting from the provision of said one or more preceding sets of electrical stimulation;

recording the said subsequent set of stimulation in a recording means;

comparing the recorded said one or more sets of electrical stimulation with the subsequent set of electrical stimulation; and

outputting an indication signal indicative of variation in the electrical stimulation between the sets to achieve the substantially equivalent movement of said body portion.

In this aspect, the step of monitoring the movement of said body portion comprises measuring the magnitude of the movement of said body portion. The step of adjusting said subsequent set of electrical stimulation preferably results in an equivalent movement magnitude of said body portion compared to that resulting from the provision of said one or more sets of electrical stimulation.

In one embodiment of this aspect, the step of recording said one or more sets of electrical stimulation or the subsequent set of electrical stimulation comprises recording the magnitude of the electrical stimulation, such as the amplitude of the stimulation impulses.

Still further, the indication signal can be used as a muscle fatigue warning signal if the magnitude of the subsequent set of electrical stimulation required to achieve the equivalent movement of said body portion increases above a predetermined level in comparison to that recorded for said one or more sets of electrical stimulation, such as when the muscles of said body portion were unfatigued. In one embodiment, this method can include a step of preventing electrical stimulation to at least said body portion if a muscle fatigue warning signal is generated.

### BRIEF DESCRIPTION OF THE DRAWINGS

By way of example only, a preferred embodiment of the invention is now described with reference to the accompanying drawings, in which:

FIG. 1 is a view of a subject walking through use of a functional electrical stimulation (FES) system according to the present invention; and

FIG. 2 is a block diagram of one embodiment of the functional electrical stimulation system according to the present invention.

### PREFERRED MODE OF CARRYING OUT THE INVENTION

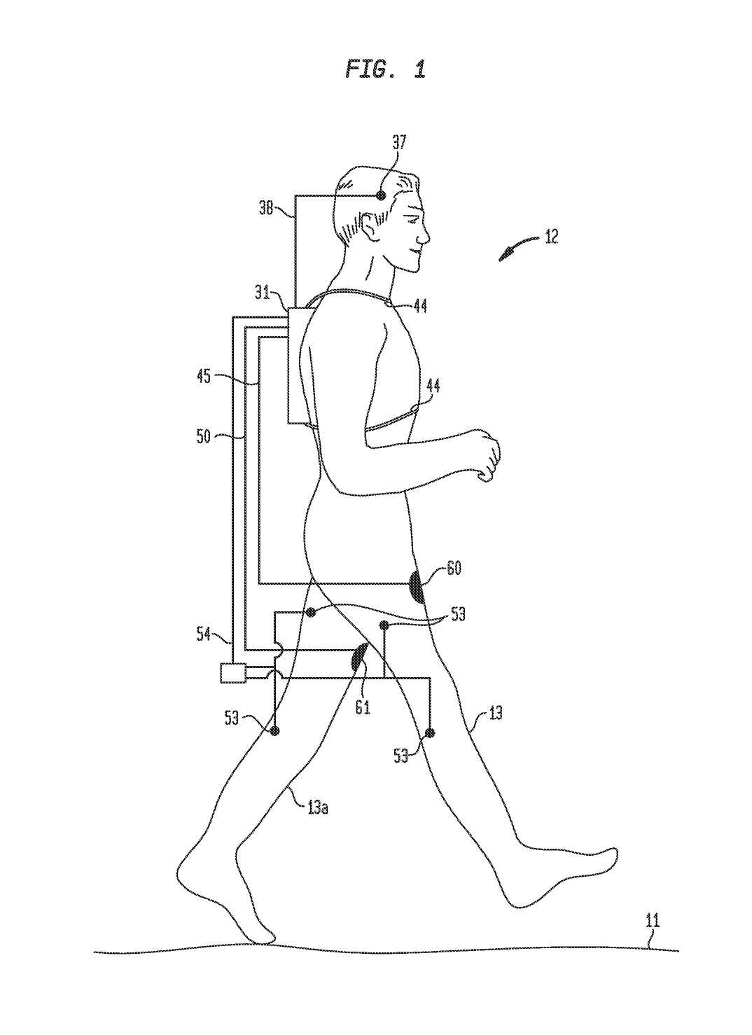

In FIG. 1, a person 12 suffering from spinal cord injury or deficiency is shown walking across a surface 11 through use of functional electrical stimulation supplied to the legs 13,13a of the person 12.

While the person 12 is shown using functional electrical stimulation to walk, it will be appreciated that this is only depicted for illustrative purposes. The person 12 could instead be using functional electrical stimulation to ride a bicycle, such as an exercise bicycle, stand, row or otherwise move their body or a normally dysfunctional portion thereof.

In FIG. 1, a functional electrical system 30 having a housing 31 is shown strapped by straps 44 to the body of the person 12. The housing 31 is used to protect the componentry of the functional electrical system 30. The housing 31 is not to be regarded as depicted to scale in FIG. 1. While shown strapped to the back of the person 12 in FIG. 1, the housing 31 could be mounted on another location, such as in the person's clothing. Yet still further, some of the componentry within the housing 31 could be implanted within the person 12.

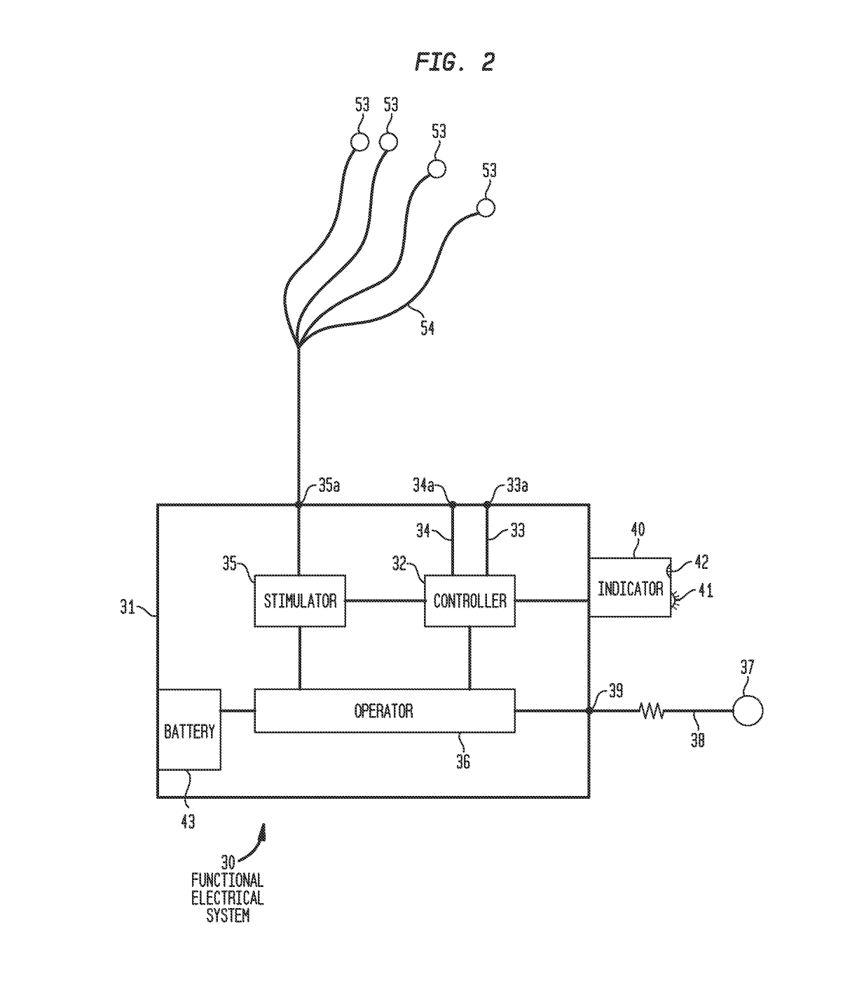

In the arrangement depicted in FIG. 2, the housing 31 can be seen to house a control means 32. A power source in the form of a rechargeable battery 43 is also provided in the housing 31. The depicted control means 32 receives a first set of signals through a first signal path 33 provided by a cable 40 extending from a first transducer 60 mounted to the thigh of one leg 13 of the person 12 to an electrical connector 33a on the housing 31. The signals to output by transducer 60 and provided through signal path 33 represent the magnitude of movement of the leg 13 as the person 12 walks across surface 11.

The control means 32 also receives a second set of output signals through a second signal path 34 provided by a cable 50 extending from a transducer 61 mounted to the other thigh of the person 12 to a connector 34a on the housing 31. While depicted schematically in FIG. 1, the cables 40,50 can be envisaged as being a flexible cables extending between the respective transducers 60,61 and the connectors 33a,34a of the housing 31. The signals output by transducer 61 and provided through signal path 34 represent the magnitude of movement of the other leg 13a as the person 12 walks across surface 11.

The control means 32 is programmed to receive the signals fed by signal paths 33 and 34 and then output a suitable sequence of signals to the stimulator 35. In this way, the control means 32 is able to output a stimulation pattern that sets the legs 13,13a moving in such a way that the person 12 can walk across the surface 11. In this regard, it will be appreciated that the stimulation sequence to say leg 13 will be 180c out of phase to the stimulation sequence provided to the person's other leg 13a.

While in the depicted embodiment, a single transducer is shown mounted to the thigh of each of the legs 13,13a of the person 12, it will be appreciated that more than one transducer or inertial sensor could be mounted on the person 12 in other positions. For examples each leg could have more than one transducer mounted thereto. Still further, one or more transducers could be mounted to the torso and/or head of the person 12.

The control means 32 comprises a microprocessor and includes a data storage device that stores measured leg movement magnitude measured by the transducers 60,61 resulting from the provision of electrical stimulation thereto.

In FIG. 2, the control means 32 includes a comparator that can compare different variables and, if necessary, so cause the stimulator 35 to modify the stimulation output to the person 12.

In one arrangement, the comparator can compare various stored measured leg movement magnitudes. The comparator can compare measured movement magnitudes of one or both of the legs 13,13a. In this arrangement, if the comparator detects a decrease in leg movement magnitude over time, this is, in the depicted embodiment, interpreted by the control means 32 as resulting from muscle fatigue within that limb. For example, if the stride of one or both of the legs 13,13a is determined by the comparator within the control means 32 as having decreased compared to that measured earlier by the transducers 60,61, this can be taken as resulting from muscle fatigue within that leg.

If the leg movement magnitude falls below a predetermined level in comparison to that earlier measured by the transducers 60,61, the control means 32 can output a warning signal to a indicator device 40 mounted to the housing 31. As depicted, the indicator device 40 can have a warning light 41 and a buzzer 42 that can be activated by the control means 32 to indicate to the person 12 that their stimulated limbs have reached a certain level of fatigue. The person 12 can be trained to recognise and heed activation of the indicator device 40 and so ensure that the system 30 does not continue to stimulate their legs 13,13a to a degree beyond that considered appropriate.

In an alternative arrangement, the control means 32 could, on or some time after activating the warning device 40, shut down the stimulator 35 and ensure the stimulator 35 does not operate until a minimum predetermined rest time had passed.

On determining that there is a decrease in leg movement magnitude, the control means 32 can also be programmed to increase the level of stimulation output by the stimulator 35, with the comparator then comparing the variation in leg movement resulting from the increase in stimulation and the control means 32 storing the amount of electrical energy required for the stimulation. If the resulting leg movement is too great, the control means 32 can decrease the level of stimulation output by the stimulator 35. The feedback provided by the transducers 60,61 serves to ensure that the system 30 continuously provides appropriate levels of stimulation whilst ensuring that the muscles of the person's leg are not overfatigued.

In another arrangement, the comparator can as well or instead undertake a comparison of the levels of stimulation output by the stimulator 35, under the control of the control means 32, to achieve equivalent or substantially equivalent degrees of leg movement magnitude. This comparison can also be used as a measure of muscle fatigue as any increase in stimulation, or sustained increase in stimulation, is considered indicative that the muscles are becoming fatigued.

In use, the stimulator 35 has a maximum safe level of stimulation that can be output to the legs 13,13a. This ensures that a safe level of stimulation is not provided to the legs despite the control means 32 noting a decrease in movement magnitude of the legs 13,13a.

Prior to use of the system 30, the person 12 themselves or a third party can connect the transducers 60,61 to their thighs. An appropriate number of stimulating electrodes 53 are also mounted to the legs of the person 12. The electrodes 53 receive stimulation pulses via cables 54 connected to the stimulator 35 by the connector 35a in the housing 31. More or less electrodes than that depicted can be envisaged depending on the requirements of the device. The length of the cables 54 depicted in FIG. 2 are also not necessarily to scale. More than one electrode per cable 54 may also be envisaged as being encompassed within this description. While the depicted arrangement relies on use of external electrodes, it should be appreciated that electrodes could be implanted within the person 12 with stimulation pulses being provided from a stimulator also implanted within the body of the person 12. Such an implanted stimulator could be used in association with an external device that communicates with the stimulator and receives control signals from the control means 32. For example, radio frequency (RF) transmission could be used to deliver signals from the external device to the implanted stimulator.

The transducers 60,61 mounted to the thighs of the person 12 provide respective signal outputs representative of the angle of the respective thighs relative to a notional plane, such as a vertical or horizontal plane. From a determination of this angle, the magnitude of the stride of the person 12 can be determined and measured.

The transducers 60,61 mounted to the legs also provide signal outputs representative of the movement of the legs following the provision of electrical stimulation to the legs by the stimulator 35. For example, the transducers 60,61 provide outputs that inform the control means 32 that the stimulator 35 has or has not achieved the outcome for the legs expected by the provided stimulation.

As depicted in FIG. 2, the system 30 further comprises an operating means 36 that receives signals from a transducer 37 adapted to monitor the position of a portion of the subject's body other than the monitored limb. The transducer 37 outputs signals through cable 38 connected to connector 39 in the housing 31. The transducer 37 and cable 38 are not depicted to scale. More than one such transducer 37 can also be envisaged. On receipt of a predetermined signal from the transducer 37, the operating means can activate or deactivate the control means 32 and/or the stimulator 35. For example, the transducer 37 can be mounted to the head of the person 12, and adapted to output a predetermined signal on determination of a particular movement of the person's head relative to the their torso. This provides the person 12 with a ready means to activate or deactivate the FES system 30 simply by a predetermined movement of their head. It can be envisaged that movement of the subject's torso or a walking aid held by the person 12 could also be used to control the signals being output to the operating means 36.

Some or all of the components of the depicted FES system 30 can be fully implanted within the person 12. It will, however, be appreciated that the control means 32 and other components could be external the body of the person 12. Electrical stimulation to the muscles is provided, in the depicted embodiment, by electrodes to the muscles identified as requiring stimulation to achieve the movement desired when installing the system 30.

The present system 30 provides a means of warning paralysed persons relying on FES systems for mobility of muscle fatigue before their stimulated muscles fail. It is anticipated that this feature will increase the confidence of persons that the system will not harm them unknowingly and so increase the likelihood of use of such systems by paralysed persons or others with impaired mobility.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1-52. (canceled)

53. A functional electrical stimulation system comprising:
a stimulator configured to generate first and second substantially equivalent sets of functional electrical stimulation for delivery to a portion of a subject's body;
a transducer mountable to said body portion and configured to measure the magnitude of movement of said body portion in response to each of said first and second sets of functional electrical stimulation and to output first and second movement magnitude signals representative of first and second measured magnitudes of movement in response to said first and second sets of functional electrical stimulation, respectively; and
a controller configured to receive the first and second movement magnitude signals and store the first and second magnitudes;
wherein the controller is configured to compare the first and second magnitudes to one another, and to provide an indication of muscle fatigue of said body portion when the second magnitude is below the first magnitude by a predetermined amount.

54. The system of claim 53, wherein the transducer is mountable to at least one of the legs of the subject.

55. The system of claim 53, wherein the transducer is implantable within the subject.

56. The system of claim 53, wherein the controller includes a memory, and wherein the controller is configured to store the first and second magnitudes in the memory.

57. The system of claim 53, wherein the portion of the subject's body is a limb of the subject.

58. The system of claim 53, wherein the controller is configured to output the indication when the second magnitude is less than about 90% of the first magnitude.

59. The system of claim 53, wherein the controller is configured to output the indication when the second magnitude is less than about 75% of the first magnitude.

60. The system of claim 53, wherein the indication is output to an indicator device.

61. The system of claim 60 wherein the indicator device is configured to provide at least one of a visual and an audible output in response to the warning signal.

62. The system of claim 53, further comprising:
an overrider configured to prevent delivery of electrical stimulation to said body portion in response to said indication.

63. The system of claim 53, wherein the controller is adapted to increase the level of functional electrical stimulation provided to said body portion if the second magnitude is below the first magnitude.

64. The system of claim 63 wherein the stimulator has a maximum stimulation level.

65. The system of claim 53, wherein the stimulator comprises one or more electrodes mountable on said body portion and electrically connected to the output of the stimulator.

66. The system of claim 53, wherein each set of functional electrical stimulation comprises one or more individual stimulation impulses.

67. The system of claim 66 wherein each set of functional electrical stimulation comprises a predetermined sequence of individual impulses suitable for moving said body portion in a desired manner.

68. A method of providing functional electrical stimulation to a portion of a subject's body and detecting muscle fatigue in the body portion, the method comprising:
providing first and second substantially equivalent sets of functional electrical stimulation to a plurality of muscles of said body portion;
measuring the magnitude of movement of said body portion in response to each of said first and second sets of functional electrical stimulation;
outputting first and second movement magnitude signals representative of first and second measured magnitudes of movement in response to said first and second sets of functional electrical stimulation, respectively;
storing the first and second magnitudes;
comparing the first and second magnitudes to one another;
outputting an indication signal indicative of muscle fatigue of said body portion when the second magnitude is below the first magnitude by a predetermined amount; and
preventing, in response to said indication signal, stimulation of only a subset of said plurality of muscles.

69. The method of claim 68, further comprising:
storing the storable movement magnitude signals.

70. The method of claim 68, further comprising:
preventing electrical stimulation to at least said body portion if said indication signal is generated.

71. A functional electrical stimulation system comprising:
a stimulator configured to generate first and second substantially equivalent sets of functional electrical stimulation for delivery to a plurality of muscles of a portion of a subject's body;
a transducer mountable to said body portion and configured to measure the magnitude of movement of said body portion in response to each of said first and second sets of functional electrical stimulation and to output first and second movement magnitude signals representative of first and second measured magnitudes of movement in response to said first and second sets of functional electrical stimulation, respectively;
a controller configured to receive the first and second movement magnitude signals, store the first and second magnitudes, wherein the controller to is configured to compare respective stimulation levels of the first and second sets of functional electrical stimulation and provide an indication of muscle fatigue of said body portion when the first and second magnitudes are substantially equivalent and the stimulation level of the second set is greater than the stimulation level of the first set by a predetermined amount; and
an overrider configured to, in response to the indication, prevent stimulation of only a subset of said plurality of muscles.

72. The system of claim 71, wherein the controller is further configured to determine an amount of muscle fatigue based on a difference between the respective stimulation levels of the first and second sets of functional electrical stimulation.

73. The system of claim 71, wherein said overrider is configured to prevent stimulation of said subset of said plurality of muscles only for a predetermined period of time.

74. The system of claim 71, wherein said subset includes muscles determined by the controller to be fatigued.

75. The system of claim 53, wherein the system is configured to deliver said first and second sets of functional electrical stimulation to a plurality of muscles of said body portion, the system further comprising:
an overrider configured to, in response to said indication, prevent stimulation of only a subset of said plurality of muscles.

76. The system of claim 53, wherein the first and second sets of functional electrical stimulation are the same.

77. The method of claim 68, wherein the first and second sets of functional electrical stimulation are the same.

78. The system of claim 71, wherein the first and second sets of functional electrical stimulation are the same.
